(19) United States
(12) Patent Application Publication
(10) Pub. No.: US 2006/0211749 A1
(43) Pub. Date: Sep. 21, 2006

(54) Treatment with omega-3 fatty acids and PPAR agonist and/or antagonist and a combination product thereof

(76) Inventors: George Bobotas, Tarpon Springs, FL (US); Robert A. Shalwitz, Bexley, OH (US); Roelof M.L. Rongen, Califon, NJ (US)

Correspondence Address: ARENT FOX PLLC, 1050 CONNECTICUT AVENUE, N.W., SUITE 400, WASHINGTON, DC, 20036, US

(21) Appl. No.: 11/293,513

(22) Filed: Dec. 5, 2005

Related U.S. Application Data
(60) Provisional application No. 60/633,125, filed on Dec. 6, 2004.

(51) Int. Cl. A61K 31/426; A61K 31/22; A61K 31/202; A61K 31/192

(52) U.S. Cl. 514/369; 514/571; 514/560; 514/547

(57) ABSTRACT

A method and composition for blood lipid therapy that comprises administering to the subject an effective amount of a PPAR agonist and/or antagonist and an omega-3 fatty acid. The methods and compositions include combination products or concomitant therapy for the treatment of subjects with hypertriglyceridemia, hypercholesteremia, mixed dyslipidemia, vascular disease, artherosclerotic disease and related conditions, obesity, the prevention or reduction of cardiovascular and vascular events, the reduction of insulin resistance, fasting glucose levels and postprandial glucose levels, and/or the reduction of incidence and/or the delay of onset of diabetes.

The present application claims priority from provisional patent application Ser. No. 60/633,125, filed Dec 6, 2004. The disclosure of the provisional application is hereby incorporated by reference.

### FIELD OF THE INVENTION

The present invention relates to a method utilizing a PPAR agonist and/or antagonist and omega-3 fatty acids for the treatment of subjects with hypertriglyceridemia, hypercholesteremia, mixed dyslipidemia, vascular disease, artherosclerotic disease and related conditions, obesity, the prevention or reduction of cardiovascular and vascular events, the reduction of insulin resistance, fasting glucose levels and postprandial glucose levels, and/or the reduction of incidence and/or the delay of onset of diabetes. The present invention also relates to a combination product of PPAR agonist and/or antagonist and omega-3 fatty acids.

### BACKGROUND OF THE INVENTION

In humans, cholesterol and triglycerides are part of lipoprotein complexes in the bloodstream, and can be separated via ultracentrifugation into high-density lipoprotein (HDL), intermediate-density lipoprotein (IDL), low-density lipoprotein (LDL) and very-low-density lipoprotein (VLDL) fractions. Cholesterol and triglycerides are synthesized in the liver, incorporated into VLDL, and released into the plasma. High levels of total cholesterol (total-C), LDL-C, and apolipoprotein B (a membrane complex for LDL-C) promote human atherosclerosis and decreased levels of HDL-C and its transport complex, apolipoprotein A, which are associated with the development of atherosclerosis. Further, cardiovascular morbidity and mortality in humans can vary directly with the level of total-C and LDL-C and inversely with the level of HDL-C.

Fibrates such as fenofibrate, bezafibrate, clofibrate and gemfibrozil, are PPAR-alpha agonists and are used in patients to decrease lipoproteins rich in triglycerides, to increase HDL and to decrease atherogenic-dense LDL. Fibrates are typically orally administered to such patients.

Fenofibrate or 2-[4-(4-chlorobenzoyl)phenoxy]-2-methyl-propanoic acid, 1-methylethyl ester, has been known for many years as a medicinally active principle because of its efficacy in lowering blood triglyceride and cholesterol levels. Fenofibrate is very poorly soluble in water and the absorption of fenofibrate in the digestive tract is limited. Treatment with 40 to 300 mg of fenofibrate per day enables a 20 to 25% reduction of cholesterolemia and a 40 to 50% reduction of triglyceridemia to be obtained.

PPAR-gamma agonists, such as the thiazolidinediones (e.g., troglitazone, pioglitazone and rosiglitazone), have been shown to improve surrogate markers of cardiovascular risk and atherosclerosis. For example, thiazolidinediones decrease C-reactive protein and carotid intima-media thickness. Non-thiazolidinediones, such as tesaglitazar, naviglitizar and muraglitazar, are dual alpha/gamma PPAR agonists. These compounds are used for lowering glucose, insulin, triglycerides and free fatty acids.

Partial PPAR-gamma agonist/antagonists, such as metaglidasen, are used for the treatment of type II diabetes.

Marine oils, also commonly referred to as fish oils, are a good source of two omega-3 fatty acids, eicosapentaenoic acid (EPA) and docosahexaenoic acid (DHA), which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides, increase serum HDL-cholesterol, lower systolic and diastolic blood pressure and the pulse rate, and lower the activity of the blood coagulation factor VII-phospholipid complex. Further, omega-3 fatty acids seem to be well tolerated, without giving rise to any severe side effects.

One such form of omega-3 fatty acid is a concentrate of omega-3, long chain, polyunsaturated fatty acids from fish oil containing DHA and EPA and is sold under the trademark Omacor®. Such a form of omega-3 fatty acid is described, for example, in U.S. Pat. Nos. 5,502,077, 5,656,667 and 5,698,594, each incorporated herein by reference.

Subjects with mixed dyslipidemia or hypercholesteremia often present with blood levels of LDL cholesterol greater than 190 mg/dl and triglyceride levels of 200 mg/dl or higher. The use of diet and single-drug therapy does not always decrease LDL cholesterol and triglycerides adequately enough to reach targeted values in subjects with mixed dyslipidemia or hypercholesterolemia with or without a concomitant increase in triglycerides. In these subjects, a complementary combination therapy of PPAR agonist and/or antagonist and omega-3 fatty acids may be desirable.

In addition, it is known that substantial variations in a subjects circulation levels exist according to pathological conditions. In general, it is preferable to maintain the active metabolite at a level necessary to obtain the desired therapeutic effect, while absorbing the minimum amount of active principle. Consequently, it is desirable to provide formulations or treatment methods with optimized dosages that offer the highest possible bioavailability and effectiveness, while limiting any side effects.

U.S. Pat. No. 6,096,338, U.S. Pat. No. 6,267,985, U.S. Pat. No. 6,667,064, U.S. Pat. No. 6,720,001, U.S. Patent Application Publication No. 2003/0082215, U.S. Patent Application Publication No. 2004/0052824, WO 99/29300 and WO 2001/021154 disclose compositions, carrier systems and oil-in-water emulsions containing digestible oils or triglycerides with an active ingredient, such as fenofibrate.

U.S. Pat. No. 6,284,268 is directed to self-emulsifying preconcentrate pharmaceutical compositions capable of forming oil-in-water microemulsions containing omega-3 fatty acid oil and a poorly water-soluble therapeutic agent. The '268 patent formulations use a large amount of solubilizers such as surfactant (generally higher than 50% w/w, based on the weight of the solvent system) to achieve self-emulsifying compositions. For example, formulation 19 discloses a self-emulsifying preconcentrate product containing 284 mg of fish oil (about 23% w/w based on the weight of the solvent system, including the fish oil), 663 mg of a surfactant system (about 55% w/w based on the weight of the solvent system), 273 mg of a hydrophilic solvent system (about 22% w/w based on the weight of the solvent system), and 100 mg of fenofibrate. There is no disclosure in the '268 patent of a fenofibrate formulation having a solvent system based mainly on fish oil without the use of a large amount of solubilizers, such as surfactants and/or hydrophilic solvents. Nor is there any disclosure in the '268 patent regarding administration of the self-emulsifying preconcentrate fenofibrate product to subjects. Rather, the '268 patent uses fenofibrate to exemplify the solubilizing properties of the disclosed self-emulsifying compositions.

There is a need in the art for a single therapeutically effective oral dosage form comprising a combination of omega-3 fatty acids and PPAR agonist and/or antagonist that provides adequate delivery of both a therapeutically effective amount of omega-3 fatty acids and a therapeutically effective amount of PPAR agonist and/or antagonist for the treatment of subjects with hypertriglyceridemia, hypercholesteremia, mixed dyslipidemia, vascular disease, artherosclerotic disease and related conditions, obesity, the prevention or reduction of cardiovascular and vascular events, the reduction of insulin resistance, fasting glucose levels and postprandial glucose levels, and/or the reduction of incidence and/or the delay of onset of diabetes.

In the past, combinations of certain fish oils with gemfibrozil or clofibrate, have not been shown to produce any synergistic action in the treatment of hyperlipidemia and hyperlipoproteinemia. See Saify et al., Pakistan J. of Pharm. Sci. (2003) 16(2): 1-8; Pennacchiotti et al., Lipids (2001) 26(2): 121-127; Wysynski et al., Human and Experimental Toxicology (1993) 12: 337-340. In contrast, the treatment methods and combination products of the present invention comprising a PPAR agonist and/or antagonist with omega-3 fatty acids demonstrate increased effectiveness of the active ingredients. Thus, the present invention allows for a novel combination product and treatment methods with greater effectiveness.

### SUMMARY OF THE INVENTION

The present invention overcomes the above-mentioned problems, as well as others, by allowing for reduced dosages of PPAR agonist and/or antagonist and omega-3 fatty acids to provide an effective pharmaceutical treatment and minimize unwanted side effects, or by providing superior activity with “full strength” dosages of either active agent alone.

One embodiment of the present invention provides a method of utilizing a pharmaceutical composition comprising a PPAR agonist and/or antagonist and omega-3 fatty acids in the treatment of subjects with hypertriglyceridemia, hypercholesteremia, mixed dyslipidemia, vascular disease, artherosclerotic disease and related conditions, obesity, the prevention or reduction of cardiovascular and vascular events, the reduction of insulin resistance, fasting glucose levels and postprandial glucose levels, and/or the reduction of incidence and/or the delay of onset of diabetes.

Another embodiment of the present invention is a combination product, comprising a PPAR agonist and/or antagonist and omega-3 fatty acids. In one aspect of the embodiment, the combination product is used in the treatment of subjects with hypertriglyceridemia, hypercholesteremia, mixed dyslipidemia, vascular disease, artherosclerotic disease and related conditions, obesity, the prevention or reduction of cardiovascular and vascular events, the reduction of insulin resistance, fasting glucose levels and postprandial glucose levels, and/or the reduction of incidence and/or the delay of onset of diabetes.

Another subject of the invention is a method of increasing fenofibrate metabolism and/or efficacy, comprising dissolving the fenofibrate in a solvent system comprising natural or synthetic omega-3 fatty acids or pharmaceutically acceptable esters, derivatives, conjugates, precursors or salts thereof, or mixtures thereof, and thereafter administering the fenofibrate to a subject for treating hypertriglyceridemia, hypercholesteremia, mixed dyslipidemia, vascular disease, artherosclerotic disease or a condition related thereto, or obesity, or preventing or reducing a cardiovascular or vascular event, reducing insulin resistance, fasting glucose levels or postprandial glucose levels, or reducing incidence or delaying onset of diabetes in the subject.

Another subject of the invention is the use of a PPAR agonist and/or antagonist and natural or synthetic omega-3 fatty acids or pharmaceutically acceptable esters, derivatives, conjugates, precursors or salts thereof, or mixtures thereof, for the manufacture of a medicament for treating hypertriglyceridemia, hypercholesteremia, mixed dyslipidemia, vascular disease, artherosclerotic disease or a condition related thereto, or obesity, or preventing or reducing a cardiovascular or vascular event, reducing insulin resistance, fasting glucose levels or postprandial glucose levels, or reducing incidence or delaying onset of diabetes in a subject.

Other novel features and advantages of the present invention will become apparent to those skilled in the art upon examination of the following or upon learning by practice of the invention.

### BRIEF DESCRIPTION OF THE FIGURES

FIG. 1 shows the mean plasma fenofibric acid concentrations after administration of fenofibrate alone or a combination of fenofibrate and Omacor® omega-3 fatty acids.

FIG. 2 shows the mean plasma fenofibric acid concentrations after administration of a unit dose formulation of fenofibrate and omega-3 fatty acids, under both fasted and fed conditions.

### DESCRIPTION OF THE PREFERRED EMBODIMENTS

The present invention discloses the use of a PPAR agonist and/or antagonist and omega-3 fatty acids for the treatment of subjects with hypertriglyceridemia, hypercholesteremia, mixed dyslipidemia, vascular disease, artherosclerotic disease and related conditions, obesity, the prevention or reduction of cardiovascular and vascular events, the reduction of insulin resistance, fasting glucose levels and postprandial glucose levels, and/or the reduction of incidence and/or the delay of onset of diabetes, and a combination product therefor. In one embodiment, the pharmaceutical compositions of the present invention allow for improved effectiveness of each active ingredient, with one or both administered as a conventional full-strength dose, as compared to the formulations in the prior art. In another embodiment, the pharmaceutical compositions of the present invention allow for reduced dosages of PPAR agonist and/or antagonist and/or omega-3 fatty acids, as compared to the formulations in the prior art, while still maintaining or even improving the effectiveness of each active ingredient.

In preferred embodiments the pharmaceutical compositions comprise Omacor® omega-3 fatty acids, as described in U.S. Pat. Nos. 5,502,077, 5,656,667 and 5,698,594. In other preferred embodiments the pharmaceutical compositions comprise omega-3 fatty acids present in a concentration of at least 40% by weight as compared to the total fatty acid content of the composition.

In still other preferred embodiments the omega-3 fatty acids comprise at least 50% by weight of EPA and DHA as compared to the total fatty acid content of the composition, and the EPA and DHA are in a weight ratio of EPA:DHA of from 99:1 to 1:99, preferably from 1:2 to 2:1. The omega-3 fatty acids may comprise pure EPA or pure DHA.

In some embodiments of the invention, the omega-3 fatty acids are administered simultaneous to administration of the PPAR agonist and/or antagonist, e.g., as a single fixed dosage pharmaceutical composition or as separate pharmaceutical compositions administered at the same time.

In other embodiments, the administration comprises omega-3 fatty acids and a PPAR agonist and/or antagonist, wherein the omega-3 fatty acids are administered apart from the administration of the PPAR agonist and/or antagonist, but the therapy is concomitant. For example, the PPAR agonist and/or antagonist may be administered weekly with daily intake of omega-3 fatty acids, or the components can be administered at different times on the same day. One skilled in the art with the benefit of the present disclosure will understand that the precise dosage and schedule for the administration of the omega-3 fatty acids and the PPAR agonist and/or antagonist will vary depending on numerous factors, such as, for example, the route of administration and the seriousness of the condition.

The invention provides a novel method of treatment of subjects with hypertriglyceridemia, hypercholesteremia, mixed dyslipidemia, vascular disease, artherosclerotic disease and related conditions, obesity, the prevention or reduction of cardiovascular and vascular events, the reduction of insulin resistance, fasting glucose levels and postprandial glucose levels, and/or the reduction of incidence and/or the delay of onset of diabetes, comprising the administration of omega-3 fatty acids and a PPAR agonist and/or antagonist, wherein omega-3 fatty acids are administered before, simultaneous to or after administration of the PPAR agonist and/or antagonist. As noted above, the treatment of a subject with both active ingredients allows a more effective treatment from the typical dosage amount of each drug or the option of decreasing the dosage amount of each drug while maintaining an effective treatment. The administration is preferably oral administration.

The combination of a PPAR agonist and/or antagonist and omega-3 fatty acids allows for a greater effect than the additive effect expected when the two drugs are combined. Thus, the combined treatment of the two active ingredients, separately or through the novel combination product of the present invention, causes an increase in effectiveness with standard dosages or maintained effectiveness with reduced dosages of the two active ingredients. Thus, the improved bioavailability or effectiveness of the two active ingredients allows for reduction in the daily dosage amount. The side effects may also be potentially reduced as a result of the lower dosage amount.

The present invention may incorporate now known or future known PPAR agonists and/or antagonists in an amount generally recognized as safe. The term “PPAR agonists and/or antagonists” includes, but is not limited to, PPAR-alpha, PPAR-gamma, PPAR-delta, PPAR-alpha/gamma, PPAR-gamma/delta, PPAR-alpha/delta, and PPAR-alpha/gamma/delta agonists and antagonists, as well as partial agonists and/or antagonists. Specific compounds include, but are not limited to, the fibrates, the thiazolidinediones, the non-thiazolidinediones and metaglidasen. Preferably, the compound is a fibrate, such as fenofibrate, bezafibrate, clofibrate and gemfibrozil, most preferably fenofibrate.

Generally, the effect of the PPAR agonist and/or antagonist is dose dependent, i.e., the higher the dose, the greater the therapeutic affect. However, the effect of each PPAR agonist and/or antagonist is different, and therefore the level of therapeutic effect of PPAR agonist and/or antagonist cannot be necessarily be directly correlated to the level of therapeutic effects of other PPAR agonists and/or antagonists. However, those of ordinary skill in the art would understand the correct dosage to be given to a particular subject, based on experience and the seriousness of the condition.

Preferred embodiments include the administration of 300 mg or less of fenofibrate, preferably 200 mg or less, more preferably 160 mg or less, even more preferably 140 mg or less, most preferably 130 mg or less.

It is another object of the present invention to provide a combination product comprising a therapeutically effective amount of the omega-3 fatty acids and a therapeutically effective amount of fenofibrate. Because of the increased pharmaceutical effect from the treatment of a subject with the combination of active ingredients, the typical dosages of these active ingredients allows for a more effective treatment. In another embodiment, the dosage and accompanying side effects may be reduced while still maintaining an effective treatment.

As used herein, the term “omega-3 fatty acids” includes natural or synthetic omega-3 fatty acids, or pharmaceutically acceptable esters, derivatives, conjugates (see, e.g., Zaloga et al., U.S. Patent Application Publication No. 2004/0254357, and Horrobin et al., U.S. Pat. No. 6,245,811, each hereby incorporated by reference), precursors or salts thereof and mixtures thereof. Examples of omega-3 fatty acid oils include but are not limited to omega-3 polyunsaturated, long-chain fatty acids such as a eicosapentaenoic acid (EPA), docosahexaenoic acid (DHA), and α-linolenic acid; esters of omega-3 fatty acids with glycerol such as mono-, di- and triglycerides; and esters of the omega-3 fatty acids and a primary, secondary or tertiary alcohol such as fatty acid methyl esters and fatty acid ethyl esters. Preferred omega-3 fatty acid oils are long-chain fatty acids such as EPA or DHA, triglycerides thereof, ethyl esters thereof and mixtures thereof. The omega-3 fatty acids or their esters, derivatives, conjugates, precursors, salts and mixtures thereof can be used either in their pure form or as a component of an oil such as fish oil, preferably purified fish oil concentrates. Commercial examples of omega-3 fatty acids suitable for use in the invention include Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE and EPAX7010EE (Pronova Biocare a.s., 1327 Lysaker, Norway).

Preferred compositions include omega-3 fatty acids as recited in U.S. Pat. Nos. 5,502,077, 5,656,667 and 5,698,694, which are hereby incorporated herein by reference in their entireties.

Another preferred composition includes omega-3 fatty acids present in a concentration of at least 40% by weight, preferably at least 50% by weight, more preferably at least 60% by weight, still more preferably at least 70% by weight, most preferably at least 80% by weight, or even at least 90% by weight. Preferably, the omega-3 fatty acids comprise at least 50% by weight of EPA and DHA, more preferably at least 60% by weight, still more preferably at least 70% by weight, most preferably at least 80%, such as about 84% by weight. Preferably the omega-3 fatty acids comprise about 5 to about 100% by weight, more preferably about 25 to about 75% by weight, still more preferably about 40 to about 55% by weight, and most preferably about 46% by weight of EPA. Preferably the omega-3 fatty acids comprise about 5 to about 100% by weight, more preferably about 25 to about 75% by weight, still more preferably about 30 to about 60% by weight, and most preferably about 38% by weight of DHA. All percentages above are by weight as compared to the total fatty acid content in the composition, unless otherwise indicated.

The EPA:DHA ratio may be from 99:1 to 1:99, preferably 4:1 to 1:4, more preferably 3:1 to 1:3, most preferably 2:1 to 1:2. The omega-3 fatty acids may comprise pure EPA or pure DHA.

The omega-3 fatty acid composition optionally includes chemical antioxidants, such as alpha tocopherol, oils, such as soybean oil and partially hydrogenated vegetable oil, and lubricants such as fractionated coconut oil, lecithin and a mixture of the same.

The most preferred form of omega-3 fatty acids is the Omacor® omega-3 acid (K85EE, Pronova Biocare A.S., Lysaker, Norway) and preferably comprises the following characteristics (per dosage form):

| Test | Minimum Value | | Maximum Value |
|---|---|---|---|
| Eicosapentaenoic acid C20:5 | 430 | mg/g | 495 mg/g |
| Docosahexaenoic acid C22:6 | 347 | mg/g | 403 mg/g |
| EPA and DHA | 800 | mg/g | 880 mg/g |
| Total n-3 fatty acids | 90% | (w/w) | |

The combination product of a PPAR agonist and/or antagonist and concentrated omega-3 fatty acids may be administered in a capsule, a tablet, a powder that can be dispersed in a beverage, or another solid oral dosage form, a liquid, a soft gel capsule or other convenient dosage form such as oral liquid in a capsule, as known in the art. In some embodiments, the capsule comprises a hard gelatin. The combination product may also be contained in a liquid suitable for injection or infusion.

The active ingredients of the present invention may also be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use. Thus, the non-active ingredients may include coilloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide and xanthum gum.

Excipients include surfactants, such as propylene glycol monocaprylate, mixtures of glycerol and polyethylene glycol esters of long fatty acids, polyethoxylated castor oils, glycerol esters, oleoyl macrogol glycerides, propylene glycol monolaurate, propylene glycol dicaprylate/dicaprate, polyethylene-polypropylene glycol copolymer, and polyoxyethylene sorbitan monooleate, cosolvents such ethanol, glycerol, polyethylene glycol, and propylene glycol, and oils such as coconut, olive or safflower oils. The use of surfactants, cosolvents, oils or combinations thereof is generally known in the pharmaceutical arts, and as would be understood to one skilled in the art, any suitable surfactant may be used in conjunction with the present invention and embodiments thereof.

The combination product of a PPAR agonist and/or antagonist and concentrated omega-3 fatty acids is aided by the solubility of the PPAR agonist and/or antagonist in the omega-3 fatty acid oil. In the combination product, the PPAR agonist and/or antagonist is substantially dissolved in the omega-3 fatty acid oil. Thus, the combination product does not require high amounts of solubilizers, such as surfactants, cosolvents, oils or combinations thereof, to dissolve the PPAR agonist and/or antagonist. Preferably, the active ingredients are administered without the use of large amounts of solubilizers (other than the omega-3 fatty acid oil), and are substantially dissolved (i.e., less than 10%, preferably less than 5% remains undissolved in the solvent system). In preferred embodiments, if present at all, solubilizers other than the omega-3 fatty acid oil are present in amounts of less than 50% w/w based on the total weight of the solvent system in the dosage form(s), preferably less than 40%, more preferably less than 30%, even more preferably less than 20%, still more preferably less than 10% and most preferably less than 5%. In some embodiments, the solvent system contains no solubilizers other than the omega-3 fatty acid oil. As used herein, “solvent system” includes the omega-3 fatty acid oil. In other preferred embodiments, the weight ratio of omega-3 fatty acid oil to other solubilizer is at least 0.5 to 1, more preferably at least 1 to 1, even more preferably at least 5 to 1, and most preferably at least 10 to 1.

In other preferred embodiments, if present at all, the amount of hydrophilic solvent used in the solvent system is less than 20% w/w based on the total weight of the solvent system in the dosage form(s), more preferably less than 10%, and most preferably less than 5%. In certain embodiments, the amount of hydrophilic solvent used in the solvent system is between 1 and 10% w/w.

In preferred embodiments, omega-3 fatty acid oil is present in amounts of at least 30% w/w based on the total weight of the solvent system in the dosage form(s), more preferably at least 40%, even more preferably at least 50%, and most preferably at least 60%. In certain embodiments, the amount can be at least 70%, at least 80% or at least 90%.

The dosage form is stable at room temperature (about 23° C. to 27° C.) for a period of at least one month, preferably at least six months, more preferably at least one year, and most preferably at least two years. By “stable”, applicants mean that the solubilized PPAR agonist and/or antagonist does not come out of solution to any appreciable degree, for example, in amounts of less than 10%, preferably less than 5%.

The concentrated omega-3 fatty acids can be administered in a daily amount of from about 0.1 g to about 10 g, more preferably about 0.5 g to about 8 g, and most preferably from about 0.75 g to about 4 g.

The PPAR agonist and/or antagonist may be administered in an amount more than, equal to or less than the conventional full-strength dose as a single-administered product. For example, the PPAR agonist and/or antagonist may be administered in an amount of from 10-100%, preferably about 25-100%, most preferably about 50-80%, of the conventional full-strength dose as a single-administered product.

The daily dosages of PPAR agonist and/or antagonist and concentrated omega-3 fatty acids can be administered together in from 1 to 10 dosages, with the preferred number of dosages from 1 to 4 times a day, most preferred 1 to 2 times a day. The administration is preferably oral administration, although other forms of administration that provides a unit dosage of PPAR agonist and/or antagonist and concentrated omega-3 fatty acids may be used.

The present combination of a PPAR agonist and/or antagonist and concentrated omega-3 fatty acids may allow for a greater effect than any expected combined or additive effect of the two drugs alone. Moreover, the combined or additive effect of the two drugs may depend on the initial level of lipid parameter in the blood of a subject. For example, the triglyceride level of a subject is generally as normal if less than 150 mg/dL, borderline to high if within about 150-199 mg/dL, high if within about 200-499 mg/dL and very high if 500 mg/dL or higher. For any given lipid parameter, the present invention may be used to reduce the level of a “very high” down to a “high” or “borderline to high” in less than 48 weeks, preferably within 24 weeks, more preferably within 12 weeks, and most preferably within 6 weeks, 4 weeks or 2 weeks. The present invention may also be used to reduce the level of a “high” down to a “borderline to high” or “normal” in less than 48 weeks, preferably within 24 weeks, more preferably within 12 weeks, and most preferably within 6 weeks, 4 weeks or 2 weeks.

Any undesirable side effects may also be reduced as a result of the lower dosage amount and the reduction in excipients (e.g., surfactants).

All references cited herein are incorporated by reference in their entirety.

### EXAMPLES

### Example 1

The following formulations may be prepared in accordance with the invention:

Formulation 1:

| Ingredient | Mg/capsule |
|---|---|
| K85EE | 1000 |
| Fenofibrate | 32.5-100 |

Formulation 2:

| Ingredient | Mg/capsule |
|---|---|
| K80EE | 1000 |
| Dehydrated ethanol | 50 |
| Propylene glycol | 20 |
| monocaprylate | |
| Fenofibrate | 15-100 |

Formulation 3:

| Ingredient | Mg/capsule |
|---|---|
| K85EE | 1000 |
| Glycerol | 35 |
| Polyethoxylated castor | 25 |
| oil | |
| Pioglitazone | 5-50 |

Formulation 4:

| Ingredient | Mg/capsule |
|---|---|
| EPAX7010EE | 1000 |
| Propylene glycol | 30 |
| Muraglitazar | 5-50 |

### Example 2

A 51-year-old male subject was hospitalized for acute pancreatitis and diagnosed with familial hypertriglyceridemia. Upon a stringent diet and initiation of fenofibrate therapy (Antara®130 mg QD), the pancreatitis subsided and the subject was released from the hospital. However, after approximately 2 weeks of fenofibrate therapy, the triglyceride (TG) level of the subject remained 749 mg/dL. Thereafter, the subject initiated Omacor® therapy (90% omega-3 acid ethyl esters, 4 grams/day QD) while also continuing fenofibrate therapy. After one month of concomitant therapy, the subject achieved a 69% reduction in TG to 235 mg/dL. In addition, the subject achieved a 46% reduction (from 280 mg/dL to 151 mg/dL) in total cholesterol after concomitant therapy. See Table 1.

TABLE 1

| | Fenofibrate | Omacor ® + | |
|---|---|---|---|
| | alone | Fenofibrate | % Change |
| TG (mg/dL) | 749 | 235 | −69 |
| Total-Cholesterol | 280 | 151 | −46 |
| HDL-Cholesterol | 28 | 32 | +14 |
| Non-HDL-Cholesterol | 252 | 119 | −47 |
| LDL-Cholesterol | Not | 72 | NA |
| | measurable | | |

The above results demonstrate the synergistic result obtained when Omacor® and fenofibrate are administered together. These results are unexpected since subjects with TG over 500 mg/dL typically experience a decrease of only about 10% total cholesterol from administration of either agent alone. In contrast, concomitant therapy of Omacor® and fenofibrate decreased total cholesterol by an additional 30% to 35%. In addition, in such a subject group, administration of Omacor® alone is expected to reduce TG by about 45% to 50%, while the synergistic effect of combined administration of Omacor® and fenofibrate reduced TG by 69%.

### Example 3

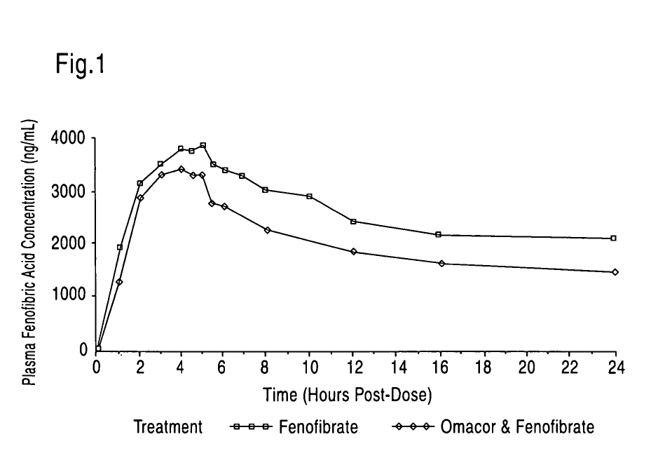

A study of 24 subjects administered with Omacor® and fenofibrate has been conducted. As shown in FIG. 1, an approximate 30% reduction of blood fenofibric acid levels (AUC) has been observed when Omacor® and fenofibrate are co-administered (circles) versus administration of fenofibrate alone (squares). The results depicted in FIG. 1 are consistent with an increased elimination constant and reduced half-life of fenofibrate when administered with Omacor® as compared to administration alone.

The observed outcome is unexpected since it is commonly known that the addition of fatty or oily substances (e.g. fatty meals) to fenofibrate enhances the blood levels of fenofibrate (AUC) (see, e.g., prescribing information for Antara® fenofibrate capsules). However, the addition of Omacor®, an oily substance consisting mostly of fatty acid esters, achieved the opposite effect. Without being limited to theory, it is believed that the observed reduction of systemic fenofibrate levels may be associated with an enhanced metabolism of fenofibrate. Thus, the present invention provides a novel method for increasing fenofibrate metabolism and/or efficacy.

### Example 4

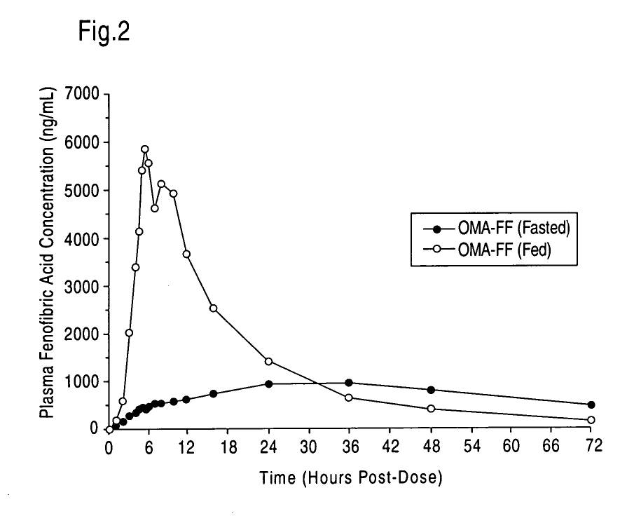

A study of patients administered a fixed dose pharmaceutical product containing fenofibrate and omega-3 fatty acids is being undertaken. In a first part of the study, the fixed dose product was administered to eight patients. FIG. 2 shows the mean fenofibric acid levels under both fed and fasted conditions.

We claim:

1. A method of treating hypertriglyceridemia, hypercholesteremia, mixed dyslipidemia, vascular disease, artherosclerotic disease or a condition related thereto, or obesity, or preventing or reducing a cardiovascular or vascular event, reducing insulin resistance, fasting glucose levels or postprandial glucose levels, or reducing incidence or delaying onset of diabetes in a subject, the method comprising administering to the subject an effective amount of a PPAR agonist and/or antagonist and natural or synthetic omega-3 fatty acids or pharmaceutically acceptable esters, derivatives, conjugates, precursors or salts thereof, or mixtures thereof.

2. The method of claim 1, wherein the PPAR agonist and/or antagonist is selected from the group consisting of a fibrate, a thiazolidinedione, a non-thiazolidinedione and metaglidasen.

3. The method of claim 1, wherein the PPAR agonist and/or antagonist comprises a fibrate.

4. The method of claim 1, wherein the PPAR agonist and/or antagonist comprises fenofibrate.

5. The method of claim 1, wherein the omega-3 fatty acids are present in a concentration of at least 40% by weight as compared to the total fatty acid content of the composition.

6. The method of claim 1, wherein the omega-3 fatty acids are present in a concentration of at least 80% by weight as compared to the total fatty acid content of the composition.

7. The method of claim 1, wherein the omega-3 fatty acids comprise at least 50% by weight of EPA and DHA as compared to the total fatty acid content of the composition.

8. The method of claim 1, wherein the omega-3 fatty acids comprise at least 80% by weight of EPA and DHA as compared to the total fatty acid content of the composition.

9. The method of claim 1, wherein the omega-3 fatty acids comprise about 5% to about 95% by weight of EPA as compared to the total fatty acid content of the composition.

10. The method of claim 1, wherein the omega-3 fatty acids comprise about 40% to about 55% by weight of EPA as compared to the total fatty acid content of the composition.

11. The method of claim 1, wherein the omega-3 fatty acids comprise about 5% to about 95% by weight of DHA as compared to the total fatty acid content of the composition.

12. The method of claim 1, wherein the omega-3 fatty acids comprise about 30% to about 60% by weight of DHA as compared to the total fatty acid content of the composition.

13. The method of claim 1, wherein omega-3 fatty acids comprise omega-3 polyunsaturated, long-chain fatty acids, esters of omega-3 fatty acids with glycerol, esters of omega-3 fatty acids and a primary, secondary or tertiary alcohol, or mixtures thereof.

14. The method of claim 1, wherein the omega-3 fatty acids comprise EPA and DHA in a ratio of EPA:DHA from 99:1 to 1:99.

15. The method of claim 1, wherein the omega-3 fatty acids comprise EPA and DHA in a ratio of EPA:DHA from 2:1 to 1:2.

16. The method of claim 1, wherein the omega-3 fatty acids are administered apart from administration of the PPAR agonist and/or antagonist.

17. The method of claim 1, wherein the omega-3 fatty acids and the PPAR agonist and/or antagonist are administered together in a unit dose form.

18. A pharmaceutical composition, comprising a PPAR agonist and/or antagonist and a solvent system comprising natural or synthetic omega-3 fatty acids or pharmaceutically acceptable esters, derivatives, conjugates, precursors or salts thereof, or mixtures thereof, wherein the omega-3 fatty acids and the PPAR agonist and/or antagonist together are present in amounts effective for treating hypertriglyceridemia, hypercholesteremia, mixed dyslipidemia, vascular disease, artherosclerotic disease or a condition related thereto, or obesity, or preventing or reducing a cardiovascular or vascular event, reducing insulin resistance, fasting glucose levels or postprandial glucose levels, or reducing incidence or delaying onset of diabetes in a subject.

19. The pharmaceutical composition of claim 18, wherein the PPAR agonist and/or antagonist is selected from the group consisting of a fibrate, a thiazolidinedione, a non-thiazolidinedione and metaglidasen.

20. The pharmaceutical composition of claim 18, wherein the PPAR agonist and/or antagonist comprises a fibrate.

21. The pharmaceutical composition of claim 18, wherein the PPAR agonist and/or antagonist comprises fenofibrate.

22. The pharmaceutical composition of claim 18, wherein the solvent system contains less than 50% w/w, based on the total weight of the solvent system, of at least one solubilizer other than the omega-3 fatty acids.

23. The pharmaceutical composition of claim 18, wherein the solvent system further comprises at least one solubilizer other than the omega-3 fatty acids in a weight ratio of omega-3 fatty acids to solubilizer of at least 0.5 to 1.

24. The pharmaceutical composition of claim 18, wherein the solvent system contains less than 20% w/w, based on the total weight of the solvent system, of at least one hydrophilic solvent.

25. The pharmaceutical composition of claim 18, wherein the pharmaceutical composition is stable for at least six months at room temperature.

25. The pharmaceutical composition of claim 18, wherein the pharmaceutical composition is in unit dose form.

26. A pharmaceutical composition in unit dose form, comprising fenofibrate and a solvent system comprising natural or synthetic omega-3 fatty acids or pharmaceutically acceptable esters, derivatives, conjugates, precursors or salts thereof, or mixtures thereof, wherein the pharmaceutical composition contains less than 50% w/w, based on the total weight of the solvent system, of at least one solubilizer other than the omega-3 fatty acids.

27. The pharmaceutical composition of claim 26, wherein the solvent system consists of the omega-3 fatty acids.

28. A method of increasing fenofibrate metabolism and/or efficacy, comprising dissolving the fenofibrate in a solvent system comprising natural or synthetic omega-3 fatty acids or pharmaceutically acceptable esters, derivatives, conjugates, precursors or salts thereof, or mixtures thereof, and thereafter administering the fenofibrate to a subject for treating hypertriglyceridemia, hypercholesteremia, mixed dyslipidemia, vascular disease, artherosclerotic disease or a condition related thereto, or obesity, or preventing or reducing a cardiovascular or vascular event, reducing insulin resistance, fasting glucose levels or postprandial glucose levels, or reducing incidence or delaying onset of diabetes in the subject.
